(19) United States
(12) Patent Application Publication
(10) Pub. No.: US 2008/0050326 A1
(43) Pub. Date: Feb. 28, 2008

(54) Deodorant composition comprising linalool and dihydromyrcenol

(76) Inventors: David Frederick Swaile, Cincinnati, OH (US); Timothy Alan Scavone, Loveland, OH (US); Barbara Marie Modafari, Mason, OH (US); Robert John Elsbrock, Cincinnati, OH (US); Lowell Alan Sanker, Cincinnati, OH (US); Jerry Anthony Janzen, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/507,856

(22) Filed: Aug. 22, 2006

(51) Int. Cl. A61K 8/31; A61K 8/18

(52) U.S. Cl. 424/65

(57) ABSTRACT

A deodorant composition comprising: a) a fragrance system comprising a fragrance material, and a combination of linalool and dihydromyrcenol, wherein the combination of linalool and dihydromyrcenol is included in the composition in an amount from about 0.2% to about 1% by weight of the composition, and wherein the ratio of linalool to dihydromyrcenol is from about 4:1 to about 1:4; and b) from about 10% to about 90%, by weight of the composition, of an emollient system comprising at least one emollient having a solubility parameter of greater than about 9 and a vapor pressure below about 2 mmHg at 25° C.

### FIELD OF THE INVENTION

The present invention is directed to deodorant compositions comprising linalool and dihydromyrcenol. The deodorant compositions may take many different product forms including, but not limited to, a solid stick, a roll-on, a liquid, and a spray product.

### BACKGROUND OF THE INVENTION

Deodorant products having high levels of perceived malodor reduction typically employ a corresponding high level of fragrance incorporation. The fragrance is generally a blend of a plethora of perfume components, each included at relatively low amounts so that individual components do not dominate the overall scent. While the high fragrance level is successful in overcoming malodor, it can manifest an overall heavy musky expression that may be considered overpowering.

Thus, there is a need for a deodorant fragrance system comprising one or more components that can be added at relatively high levels, wherein the components themselves increase the pleasantness of the scent, do not dominate the scent expression, and do not lead to an unappealing overall expression.

### SUMMARY OF THE INVENTION

The present invention is directed to deodorant compositions that provide a long-lasting, fresh pleasant scent. In accordance with one of the preferred embodiments, there has now been provided a deodorant composition comprising a fragrance system and an emollient system. The fragrance system includes a fragrance material, and a combination of linalool and dihydromyrcenol, wherein the combination of linalool and dihydromyrcenol is included in the composition in an amount from about 0.2% to about 1%, by weight of the composition, and wherein the ratio of linalool to dihydromyrcenol is from about 4:1 to about 1:4. The emollient system is included in the composition in an amount from about 10% to about 90%, by weight of the composition, and comprises at least one emollient having a solubility parameter of greater than about 9 and a vapor pressure below about 2 mmHg at 25° C.

The present invention is further directed to methods for providing a sustained scent from a deodorant composition. In accordance with one of the preferred embodiments there has now been provided a method comprising the steps of incorporating a fragrance material into the composition, and incorporating a combination of linalool and dihydromyrcenol into the composition in an amount that is greater than or equal to about 0.2% by weight of the composition.

In accordance with another preferred embodiment, there has now been provided a method comprising the steps of incorporating a fragrance material and a fragrance enhancement system into the composition. The fragrance enhancement system comprises a combination of linalool and dihydromyrcenol included in an amount that is greater than or equal to about 0.2% by weight of the composition, and an emollient for solubilizing the linalool and dihydromyrcenol.

### DETAILED DESCRIPTION OF THE INVENTION

Applicant has unexpectedly discovered that employment in deodorant compositions of two specific perfume ingredients—linalool and dihydromyrcenol—at relatively high levels can result in a long lasting fresh and pleasant scent that is not overpowering, but yet provides excellent perceived malodor protection. The levels of incorporation are higher than that typically chosen by the skilled artisan. Applicant has also discovered that employment of a moderately volatile to nonvolatile polar organic or organosilicone emollient with a solubility parameter greater than 9 can enhance the long lasting fresh scent provided by the specific combination of linalool and dihydromyrcenol. The deodorant compositions of the present invention may be formulated as an aqueous or anhydrous composition. Exemplary aqueous compositions may include from about 10% to about 60% water, by weight of the composition. Exemplary anhydrous compositions can include from less than about 10%, less than about 3%, less than about 1%, or zero percent water, by weight of the composition.

The present invention may be understood more readily by reference to the following detailed description of illustrative and preferred embodiments. It is to be understood that the scope of the claims is not limited to the specific ingredients, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values form another embodiment. All ranges are inclusive and combinable.

All percentages and ratios used herein are by weight of the total composition, and all measurements made are at 25° C., unless otherwise designated.

The compositions of the present invention can comprise, consist of, and consist essentially of the elements and limitations of the invention described herein, as well as any of the additional or optional ingredients, components, steps, or limitations described herein.

The term “ambient conditions” as used herein refers to surrounding conditions at about one atmosphere of pressure, about 50% relative humidity and about 25° C.

The term “volatile” as used herein refers to those materials which have a measurable vapor pressure as measured at 25° C. and 1 atmosphere. The term “moderately volatile material,” as used herein, refers to those materials with a vapor pressure below about 2 mmHg at 25° C. The term “low volatile material,” as used herein, refers to those materials with a vapor pressure below about 0.5 mmHg at 25° C. The term “nonvolatile material,” as used herein, refers to those materials with a vapor pressure below about 0.002 mmHg at 25° C. Vapor pressures can be measured in a variety of manners and are often available in a variety of chemical data bases that would be known to one skilled in the art. One such database is available from the Research Institute for Fragrance Materials.

The solubility parameters for various solvents or other materials described herein are determined by methods well known in the chemical arts. A description of solubility parameters and means for determining them are described by C. D. Vaughan, “Solubility: Effects in Product, Package, Penetration and Preservation”, 103 Cosmetics and Toiletries 47-69, October 1988 and C. D. Vaughan, “Using Solubility Parameters in Cosmetics Formulation”, 36, J. Soc. Cosmetic Chemists 319-333, September/October, 1985.

### I. EXEMPLARY DEODORANT COMPOSITIONS

Deodorant compositions of the present invention include a fragrance system and an emollient system, and may contain any number of optional ingredients that are described below and/or known to the skilled artisan.

A. Fragrance System

The fragrance system employed by embodiments of the present invention includes a combination of linalool and dihydromyrcenol, and one or more fragrance materials. Applicants have surprisingly discovered that linalool and dihydromyrcenol can be added at relatively high levels, but yet not strike through the overall scent in an overpowering manner. As a combination, these two materials are preferably added in the range of about 0.1% to about 1%, by weight of the complete deodorant composition. The combined level may be lower than 0.1% and may be higher than 1% depending on the application and/or nature of the additional materials included in the overall composition. In one preferred embodiment, the combination of linalool and dihydromyrcenol is added in an amount of from about 0.2% to about 0.6%, by weight of the deodorant composition. Preferred ratios of linalool to dihydromyrcenol include from 4:1 to 1:4, and more preferred from 2:1 to 1:2.

One or more fragrance materials are included to help cover or mask malodors resulting from perspiration or which otherwise provide the compositions with the desired perfume aroma. These fragrance materials may include any perfume or perfume chemical suitable for topical application to the skin.

The concentration of the fragrance in the deodorant compositions should be effective to provide the desired aroma characteristics or to mask malodor wherein the malodor is inherently associated with the composition itself or is associated with malodor development from human perspiration. Deodorant compositions of the present invention may comprise fragrances selected from the group consisting of free perfumes, encapsulated perfumes, and mixtures thereof. The total perfume may include one or more individual perfume chemicals provided that the perfume can emit a detectable perfume odor or can mask or help to mask odors associated with perspiration. Generally, the deodorant compositions of the present invention may comprise the total perfume at concentrations ranging from about 0.05% to about 10%, preferably from about 0.5 to about 5% and more preferably from about 1 to about 4%.

Nonlimiting examples of fragrance materials suitable for use as a free perfume or an encapsulated perfume include any known fragrances in the art or any otherwise effective fragrance materials. Typical fragrances are described in Arctander, Perfume and Flavour Chemicals (Aroma Chemicals), Vol. I and II (1969) and Arctander, Perfume and Flavour Materials of Natural Origin (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314); aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Suitable fragrance materials may also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the present invention may comprise fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof.

B. Emollient System

Deodorant compositions of the present invention comprise an emollient system including at least one, and preferably a combination of different emollients. The emollients are preferably liquid under ambient conditions, and comprise at least one emollient having a solubility parameter greater than about 9 and more preferably greater than about 11, and a vapor pressure below about 2 mm Hg at 25° C. Depending on the type of product form desired, concentrations of the emollient(s) in the deodorant compositions can range from about 10% to about 90%, and preferably from about 25% to about 75%, by weight of the deodorant composition.

Emollients suitable for use in the deodorant compositions of the present invention include, but are not limited to, topically safe and effective organic, silicone-containing or fluorine-containing, volatile or non-volatile, polar or non-polar, provided that the resulting combinations of emollients form a solution or other homogenous liquid or liquid dispersion at the selected processing temperature of the composition. Processing temperatures for the deodorant compositions can range from about 15° C. to about 150° C., depending on product form. Process temperatures for stick deodorant products typically range from about 50° C. to about 100° C. Preferred emollients with the requisite solubility parameter and volatility include but are not limited to: propylene glycol, dipropylene glycol, tripropylene glycol, diethylene glycol, triethylene glycol, PEG-4, PEG-8, 1,2 pentanediol, 1,2 hexanediol, hexylene glycol, and glycerin. Applicant has discovered that, in some product forms, blends of these emollients are most preferred.

Other emollients can be included in the product to provide other benefits to the deodorant such as, for example, good skin feel or skin moisturization. Other examples of suitable emollients include C2 to C20 monohydric alcohols, C2 to C40 dihydric or polyhydric alcohols, alkyl ethers of polyhydric and monohydric alcohols, volatile silicone emollients such as cyclopentasiloxane, non volatile silicone emollients such as dimethicone, mineral oils, polydecenes, and petrolatum.

C. Thickening or Structuring Agent

Deodorant compositions of the present invention may comprise a suitable thickening or structuring agent in order to provide the desired hardness and application characteristics to the compositions. The thickening or structuring agent concentrations may range from about 0.1%, to about 30%, by weight of the composition depending on the type of product and thickening or structuring agent. Preferred inclusion ranges for stick products are from about 4 to about 20%.

Aqueous deodorant compositions of the present invention may comprise a thickening or structuring agent that can melt to form a solution or other homogenous liquid or liquid dispersion within the liquid carrier at a processing temperature of from about 50° C. or to about 150° C., preferably from about 60° C. to about 100° C.

Suitable thickening or structuring agents for use in the aqueous and anhydrous deodorant compositions of the present invention include, but are not limited to, fatty acid gellants, salts of fatty acids, hydroxy fatty acid gellants, esters and amides of fatty acid or hydroxy fatty acid gellants, cholesterolic materials, dibenzylidene alditols, lanolinolic materials, fatty alcohols, triglycerides, and other suitable gellants. Other examples include finely divided or colloidal silicas, fumed silicas, and silicates, which includes montmorillonite clays and hydrophobically treated montmorillonites, e.g., bentonites, hectorites and colloidal magnesium silicates.

Preferred thickening or structuring agents for use in the aqueous and anhydrous deodorant compositions are the solid salts of fatty acids wherein the fatty acid moiety has from about 12, from about 16 or from about 18 carbon atoms to about 40, to about 22, or about 20 carbon atoms. Suitable salt forming cations for use with these thickening or structuring agents include metal salts such as alkali metals (e.g. sodium and potassium), alkaline earth metals (e.g. magnesium), and aluminum. Preferred are sodium and potassium salts. For example, suitable salt forming cations may be selected from the group consisting of sodium stearate, sodium palmitate, potassium stearate, potassium palmitate, sodium myristate, aluminum monostearate, and combinations thereof.

D. Propellants

Depending on the chosen product form, the deodorant compositions of the present invention may employ a propellant. Suitable propellants include, but are not limited to, dimethylether, 1,1 difluoroethane, 1,1,1,2 tetrafluoro ethane, carbon dioxide, butane, isobutane, propane, isopentane, pentane, nitrous oxide, carbon dioxide, halogenated hydrocarbons such as trichlorofluoromethane, dichlorodifluoromethane, dichlorotetrafluoroethane trichlorotrifluoroethane, trichlorotetrafluoroethane, and monochlorodifluoromethane, and combinations thereof. The total propellant concentration in the deodorant compositions can range from about 5% to about 99%, more typically from about 15% to about 90%, even more preferably from about 20% to about 70%, by weight of the composition.

E. Optional Ingredients

In addition to the aforementioned components, the deodorant compositions of the present invention may further comprise one or more optional components which may modify the physical or chemical characteristics of the compositions or serve as additional “active” components when deposited on the skin. Of course, such optional components may be included provided that they are physically and chemically compatible and do not otherwise unduly impair product stability, aesthetics, or performance. Nonlimiting examples of such optional materials include, but are not limited to, pH buffering agents, additional malodor controlling agents such as deodorant actives, fragrance materials, emollients, humectants, soothing agents, dyes and pigments, medicaments, baking soda and related materials, preservatives, and soothing agents such as aloe vera, allantoin, D-panthenol, avocado oil and other vegetative oils, and lichen extract.

Suitable optional deodorant actives may include any topical material that is known or otherwise effective in preventing or eliminating malodor associated with perspiration. Suitable deodorant actives may be selected from the group consisting of antimicrobial agents (e.g., bactericides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. The concentration of the optional deodorant active may range from about 0.001%, from about 0.01%, of from about 0.1%, by weight of the composition to about 20%, to about 10%, to about 5%, or to about 1%, by weight of the composition.

### II. METHOD OF USE

The deodorant compositions of the present invention may be topically applied to the axilla or other area of the skin in any known or otherwise effective method for controlling malodor associated with perspiration. These methods comprise applying to the axilla or other area of the human skin a safe and effective amount of the deodorant composition of the present invention. “Safe and effective amount” means an amount of the deodorant composition topically applied to the skin which is effective in inhibiting or minimizing or masking perspiration malodor at the site of application while also being safe for human use at a reasonable risk/benefit ratio. Thus, a safe and effective amount, as used in the present invention may for example range from about 0.1 gram per axilla to about 2.0 gram per axilla. The compositions are preferably applied to the axilla or other area of the skin one or more times daily, preferably once daily.

### III. EXAMPLES

Deodorant Composition Examples A-E. The numerical values in the table are in percent by weight of the total composition.

| Ingredient | A | B | C | D | E |
|---|---|---|---|---|---|
| Product Form | Solid | Solid | Solid | Solid | Deodorant |
| | Deo- | Deo- | Deo- | Deodorant | or Body |
| | dorant | dorant | dorant | | Spray |
| dipropylene glycol | 45 | 22 | 20 | 30 | 20 |
| propylene glycol | 22 | 45 | 22 | | |
| tripopylene glycol | | | 25 | | |
| glycerine | | | | 10 | |
| PEG-8 | | | | 20 | |
| ethanol | | | | | QS |
| water | QS | QS | QS | QS | |
| sodium stearate | 5.5 | 5.5 | 5.5 | 5.5 | |
| tetra sodium EDTA | 0.05 | 0.05 | 0.05 | 0.05 | |
| sodium hydroxide | 0.04 | 0.04 | 0.04 | 0.04 | |
| triclosan | 0.3 | 0.3 | 0.3 | 0.3 | |
| fragrance | 1.5 | 1.5 | 1.5 | 1.5 | 1 |
| dihydromyrcenol | 0.3 | .1 | 0.3 | 0.5 | .1 |
| linalool | 0.2 | .15 | 0.2 | 0.25 | .15 |
| Propellant (1,1 | | | | | 40 |
| difluoroethane) | | | | | |

QS - indicates that this material is used to bring the total to 100%.

Examples A to D can be made as follows: all ingredients except the fragrance, linalool, and dihydromyrcenol are combined in a suitable container and heated to about 85° C. to form a homogenous liquid. The solution is then cooled to about 62° C. and then the fragrance, linalool, and dihydromyrcenol are added. The mixture is then poured into an appropriate container and allowed to solidify up cooling to ambient temperature.

Example E can be made as follows: all the ingredients except the propellant are combined in an appropriate aerosol container. The container is then sealed with an appropriate aerosol delivery valve. Next air in the container is removed by applying a vacuum to the valve and then propellant is added to container through the valve. Finally an appropriate actuator is connected to the valve to allow dispensing of the product.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A deodorant composition, comprising:
a) a fragrance system comprising a fragrance material, and a combination of linalool and dihydromyrcenol, wherein the combination of linalool and dihydromyrcenol is included in the composition in an amount from about 0.2% to about 1% by weight of the composition, and wherein the ratio of linalool to dihydromyrcenol is from about 4:1 to about 1:4; and
b) from about 10% to about 90%, by weight of the composition, of an emollient system comprising at least one emollient having a solubility parameter of greater than about 9 and a vapor pressure below about 2 mmHg at 25° C.

2. The deodorant composition of claim 1, wherein the combination of linalool and dihydromyrcenol is included in the composition in an amount from about 0.25% to about 0.6% by weight of the composition.

3. The deodorant composition of claim 1, wherein the linalool is included in an amount from about 0.15% to about 0.25% by weight of the composition.

4. The deodorant composition of claim 1, wherein the hydromyrcenol is included in an amount from about 0.1% to about 0.5% by weight of the composition.

5. The deodorant composition of claim 1, wherein the emollient system is included in an amount from about 50% to about 90% by weight of the composition.

6. The deodorant composition of claim 5, wherein the emollient system is included in an amount from about 60% to about 70% by weight of the composition.

7. The deodorant composition of claim 1, wherein the at least one emollient has a solubility parameter of greater than about 11.

8. The deodorant composition of claim 1, wherein the emollient system comprises materials selected from the group consisting of propylene glycol, dipropylene glycol, tripropylene glycol, polypropylene glycol, polyethylene glycol, and combinations thereof.

9. The deodorant composition of claim 1, wherein the composition is in the form of a solid deodorant.

10. A method for providing a sustained scent from a deodorant composition, comprising the steps of:
a) incorporating a fragrance material into the composition; and
b) incorporating a combination of linalool and dihydromyrcenol into the composition in an amount that is greater than or equal to about 0.2% by weight of the composition.

11. The method of claim 10, wherein the combination of linalool and dihydromyrcenol is included in an amount that is greater than or equal to about 0.5% by weight of the composition.

12. The method of claim 10, wherein the linalool is included in an amount that is greater than or equal to about 0.15% by weight of the composition.

13. The method of claim 10, wherein the dihydromyrcenol is included in an amount that is greater than or equal to about 0.1% by weight of the composition.

14. A method for providing a sustained scent from a deodorant composition, comprising the steps of:
a) incorporating a fragrance material into the composition; and
b) incorporating a fragrance enhancement system into the composition, the fragrance enhancement system comprising:
i) a combination of linalool and dihydromyrcenol included in an amount that is greater than or equal to about 0.2% by weight of the composition; and
ii) an emollient for solubilizing the combination of linalool and dihydromyrcenol.

15. The method of claim 14, wherein the emollient has a solubility parameter of greater than about 9.

16. The method of claim 15, wherein the emollient has a solubility parameter of greater than about 11.

17. The method of claim 14, wherein the emollient has a vapor pressure below about 2 mmHg at 25° C.

18. The method of claim 14, wherein the emollient comprises materials selected from the group consisting of propylene glycol, dipropylene glycol, tripropylene glycol, polypropylene glycol, polyethylene glycol, and combinations thereof.

19. The method of claim 14, wherein the wherein the combination of linalool and dihydromyrcenol is included in an amount that is greater than or equal to about 0.5% by weight of the composition.
